(19) United States
(12) Patent Application Publication
(10) Pub. No.: US 2013/0137666 A1
(43) Pub. Date: May 30, 2013

(54) COMBINATIONS OF NIACIN COMPOUNDS, OMEGA-3 FATTY ACID COMPOUNDS, AND PHYTOSTEROL COMPOUNDS FOR PREVENTION AND/OR MITIGATION OF ERECTILE DYSFUNCTION

(71) Applicant: Prosoft Software, Inc., Wayne, PA (US)

(72) Inventor: Dror Rom, Wayne, PA (US)

(73) Assignee: PROSOFT SOFTWARE, INC., Wayne, PA (US)

(21) Appl. No.: 13/689,150

(22) Filed: Nov. 29, 2012

Related U.S. Application Data
(60) Provisional application No. 61/564,538, filed on Nov. 29, 2011.

(51) Int. Cl. A61K 31/56; A61K 31/455; A61K 31/202

(52) U.S. Cl. CPC A61K 31/56; A61K 31/202; A61K 31/455
USPC 514/171

(57) ABSTRACT

Provided herein are nutritional compositions, regimens and methods for the prevention, mitigation, or treatment of ED. In some embodiments, the invention comprises compositions of at least two (and preferably three) members selected from the group consisting of a niacin compound, an Omega-3 fatty acid compound, and a phytosterol compound.

### CROSS REFERENCE TO RELATED APPLICATIONS

This application claims the benefit of U.S. Provisional Patent Application No. 61/564,538, filed Nov. 29, 2011, which is incorporated by reference in its entirety as if fully set forth.

### BACKGROUND OF THE INVENTION

Erectile dysfunction (“ED”) is a recognized medical condition and disease state. The prevalence of ED in men has led to many FDA-approved new chemical entity drugs for treating ED, such as VIAGRA® brand sildenafil citrate and CIALIS® brand tadalafil, among others. ED is thought to have various causes, of which vascular efficiency is a primary factor, and vascular efficiency is known to be affected by cholesterol-induced atherosclerotic disease which narrows blood vessels. Drugs such as VIAGRA® brand sildenafil citrate and CIALIS® brand tadalafil have been shown to improve ED through a symptomatic and immediate vasodilatory effect that enhances penile blood flow, however cholesterol-induced narrowing of the blood vessels can persist in the long-term.

Cholesterol is a waxy, fat-like substance made in the liver and other cells and found in certain foods, such as food from animals, like dairy products, eggs, and meat. The body needs a limited amount of cholesterol in order to function properly (cell walls need cholesterol to produce hormones, vitamin D, and the bile acids that help to digest fat). However, excess cholesterol may lead to plaque (a thick, hard deposit) forming in the body's arteries narrowing the space for blood to flow to the heart. Over time, this buildup may cause atherosclerosis (hardening of the arteries) which can lead to multiple health problems, including ED and heart disease.

Cholesterol travels through the blood attached to a protein. The combination of the protein and the cholesterol is known as a lipoprotein. Lipoproteins are classified as high density, low density, or very low density, depending on how much protein there is in relation to fat. Very low density lipoproteins (VLDL) are similar to LDL cholesterol in that it contains mostly fat and not much protein. High density lipoproteins (HDL) also referred to as “good” cholesterol, helps the body get rid of bad cholesterol in the blood. The vast majority of scientific evidence generally supports the following: 1. The higher the level of HDL cholesterol, the better. If your levels of HDL are low, your risk of heart disease increases. 2. Low density lipoproteins (LDL) also referred to as “bad” cholesterol, can cause buildup of plaque on the walls of arteries. The more LDL there is in the blood, the greater the risk of heart disease.

Triglycerides are another type of fat that is carried in the blood by very low density lipoproteins. Excess calories, alcohol, or sugar in the body are converted into triglycerides and stored in fat cells throughout the body. Your total cholesterol is a combination of your HDL, LDL and triglycerides, such that total cholesterol=HDL+LDL+(0.2×Triglycerides).

Prevailing cholesterol guidelines define a total cholesterol number of less than 200 to be good. The guidelines also consider an HDL of greater than 40 for men and greater than 50 for women to be good. An LDL of less than 100 is ideal, while values between 130 and 159 are considered borderline and over 160 are considered high. Triglycerides of less than 150 are considered good. An LDL/HDL ratio of less than 3 is considered good and a total cholesterol/HDL ratio of less than 5 is considered good. The generally accepted cholesterol guidelines are summarized in FIG. 1.

If a person's cholesterol falls outside the cholesterol guidelines, and changing diet and/or exercise is not sufficient to bring cholesterol levels within the guidelines, medications and/or dietary supplements may be utilized to help manage cholesterol levels. Several types of prescription drugs are often used to treat cholesterol, including a class of drugs known as “statins” (e.g., rosuvastatin, fluvastatin, and atorvastatin). While statins have been shown to be quite effective in the treatment of (primarily) LDL cholesterol, they are also known or suspected of causing severe side effects. Other medications and dietary supplements are sometimes used to treat cholesterol, including: niacin (prescription or non-prescription) to increase HDL; dietary supplementation with phytosterols (e.g., plant sterols and stanols) to lower LDL; and Omega-3 fatty acids (prescription or non-prescription) to lower triglycerides. These medications and dietary supplements are considered safer by many practitioners since their side effects are generally milder compared to other classes of cholesterol-lowering prescription drugs (e.g., statins), but undesirable side effects can still occur. For example, niacin is a vasodilator and can result in skin “flushing,” where redness and an uncomfortable itching or burning sensation develop in the face, neck, and potentially other parts of the body. In addition, when these medications and dietary supplements are taken individually, their effects may be negligible or small, and insufficient when the cholesterol level is markedly out of range.

Accordingly, while drugs such as VIAGRA® brand sildenafil citrate and CIALIS® brand tadalafil exist, these drugs often cause serious side effects in some patients and there can be a high risk of drug interactions between ED drugs and the many other drugs commonly prescribed for older men. Similarly, the addition of another drug into an older human body places additional stress on body systems, such as the liver and kidneys, which must metabolize or otherwise process the drug. Lastly, it is desirable to treat potential long-term causes of ED that may persist despite acute treatment with vasodilators and other treatment options that provide transient increases in penile blood flow.

For all these reasons, there exists a continuing and unmet need for safe, effective compositions, regimens and methods for preventing, mitigating, or treating ED in men, particularly men of advanced age.

### BRIEF DESCRIPTION OF THE DRAWINGS

The features and advantages of the various embodiments will become apparent from the following detailed description in which:

FIG. 1 is a table summarizing generally accepted cholesterol guidelines.

FIG. 2 is a table summarizing dosage ranges for various cholesterol medications.

FIG. 3 illustrates an example flowchart for determining an appropriate regimen to prevent, mitigate, or treat ED in a male patient in accordance with an embodiment of the present invention.

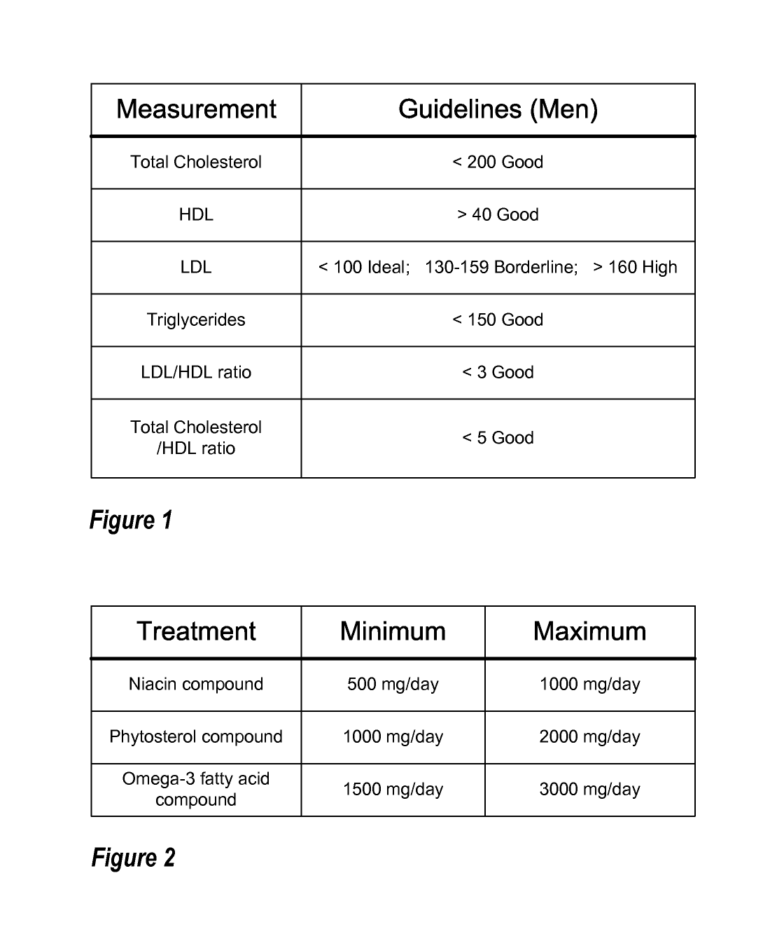

### SUMMARY OF THE INVENTION

Provided herein are nutritional compositions, regimens and methods for the prevention, mitigation, or treatment of ED.

In one embodiment, provided is a composition for the prevention, mitigation, or treatment of erectile dysfunction, comprising at least two members of the group consisting of a niacin compound, an Omega-3 fatty acid compound, and a phytosterol compound.

In another embodiment, provided is a composition for the prevention, mitigation, or treatment of erectile dysfunction, comprising a niacin compound, an Omega-3 fatty acid compound, and a phytosterol compound.

In another embodiment, provided is a composition for the prevention, mitigation, or treatment of erectile dysfunction, comprising a niacin compound, an Omega-3 fatty acid compound, and a phytosterol compound, wherein the composition comprises between 100 mg and 500 mg of the niacin compound, an Omega-3 fatty acid compound, and a phytosterol compound.

In another embodiment, provided is a composition for the prevention, mitigation, or treatment of erectile dysfunction, comprising at least two members of the group consisting of a niacin compound, an Omega-3 fatty acid compound, and a phytosterol compound, wherein the composition comprises inositol hexanicotinate.

In another embodiment, provided is a composition for the prevention, mitigation, or treatment of erectile dysfunction, comprising at least two members of the group consisting of a niacin compound, an Omega-3 fatty acid compound, and a phytosterol compound, wherein the at least two members consist of a niacin compound and one member of the group consisting of an Omega-3 fatty acid compound and a phytosterol compound.

In another embodiment, provided is a composition for the prevention, mitigation, or treatment of erectile dysfunction, comprising at least two members of the group consisting of a niacin compound, an Omega-3 fatty acid compound, and a phytosterol compound, wherein the at least two members consist of a niacin compound and one member of the group consisting of an Omega-3 fatty acid compound and a phytosterol compound, and the niacin compound and the one member of the group consisting of an Omega-3 fatty acid compound and a phytosterol are present in the composition in a 1:2 ratio, respectively.

In another embodiment, provided is a composition for the prevention, mitigation, or treatment of erectile dysfunction, comprising at least two members of the group consisting of a niacin compound, an Omega-3 fatty acid compound, and a phytosterol compound, wherein the composition does not contain any statin or other cholesterol modifying pharmaceutical.

In another embodiment, provided is a composition for the prevention, mitigation, or treatment of erectile dysfunction, comprising at least two members of the group consisting of a niacin compound, an Omega-3 fatty acid compound, and a phytosterol compound, wherein the composition does not contain any additional pharmaceutical intended to treat erectile dysfunction.

In another embodiment, provided is a composition for the prevention, mitigation, or treatment of erectile dysfunction, comprising at least two members of the group consisting of a niacin compound, an Omega-3 fatty acid compound, and a phytosterol compound, wherein composition is in an oral delivery form.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the first composition comprises a niacin compound, an Omega-3 fatty acid compound, and a phytosterol compound.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the at least two members consist of a niacin compound and one member of the group consisting of an Omega-3 fatty acid compound and a phytosterol compound.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the at least two members consist of a niacin compound and one member of the group consisting of an Omega-3 fatty acid compound and a phytosterol compound, and the niacin compound and the one member of the group consisting of an Omega-3 fatty acid compound and a phytosterol are present in the first composition in a 1:2 ratio, respectively.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; administering the first composition to the human male in a first dosage; measuring a cholesterol level in the human male; and determining that at least one compound in the first composition will positively affect the measured cholesterol level in the human male.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; administering the first composition to the human male in a first dosage; and administering the first composition to the human male in a second dosage, wherein the second dosage is greater than the first dosage.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the first composition does not contain any statin or other cholesterol modifying pharmaceutical.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the first composition does not contain any additional pharmaceutical intended to treat erectile dysfunction.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the first composition is administered to the human male in an oral delivery form.

In another embodiment, provided is a method of preventing, mitigating, or treating erectile dysfunction in a human male, the method comprising the steps of: preparing a first composition comprising at least two members of the group consisting of a niacin compound, an Omega-3 fatty acid compound, and a phytosterol compound; and administering the first composition to the human male in a first dosage, wherein the first composition comprises inositol hexanicotinate.

### DETAILED DESCRIPTION OF THE PREFERRED EMBODIMENTS

The ensuing detailed description provides preferred exemplary embodiments only, and is not intended to limit the scope, applicability, or configuration of the invention. Rather, the ensuing detailed description of the preferred exemplary embodiments will provide those skilled in the art with an enabling description for implementing the preferred exemplary embodiments of the invention. It being understood that various changes may be made in the function and arrangement of elements without departing from the spirit and scope of the invention, as set forth in the appended claims.

As used in the specification and claims, the term “niacin compound” is intended to include niacin, niacin precursors, and niacin derivatives. Niacin (also known as vitamin B₃ and nicotinic acid) is an organic compound and an essential human nutrient having the IUPAC name pyridine-3-carboxylic acid and the formula C₆H₅NO₂. Niacin is a colorless, water-soluble solid that is a derivative of pyridine, with a carboxyl group (COOH) at the 3-position. Niacin precursors are compounds that can be converted into niacin, and Niacin derivatives are compounds that are derived from niacin, such as nicotinamide (also known as niacinamide), where the carboxyl group is replaced by a carboxamide group (CONH₂), and nicotinic acid methyl ester, where the carboxyl group is replaced with a methyl ester group (CO₂CH₃). Niacin derivatives can also include more complex amides and esters, as well as compounds having other branches. Inositol hexanicotinate (IHN), which is inositol with niacin esterified on all six of inositol's alcohol groups, is a more complex derivative of niacin that is also a dietary supplement. IHN is usually sold as “flush-free” or “no-flush” niacin in tablets or capsules. As previously discussed, niacin is a vasodilator and, as such, can result in skin “flushing” where increased blood flow in the blood vessels of the face, neck and other parts of the body causes redness and an uncomfortable itching or burning sensation. “Flush-free” or “no-flush” niacin is intended to reduce flushing by releasing niacin over an extended period of time.

As used in the specification and claims, the term “phytosterol” is intended to include sterols and stanols naturally occurring in plants or those derived from a naturally occurring plant material, as well as plant sterol precursors and derivatives and plant stanol precursors and derivatives. Stanols are saturated sterols (i.e., they do not have a double bond in the sterol ring structure). The term “phytosterols” expressly includes stanol esters, which are a heterogeneous group of chemical compounds derived from phytosterols. For example, phytosterols can be first hydrogenated to give a plant stanol, which is then esterified with a fatty acid (i.e., the fatty acid, such as a fatty acid derived from plants, is attached to the stanol via an ester bond) to yield stanol esters. Plant stanol esters are also found naturally occurring in small quantities in fruits, vegetables, nuts, seeds, cereals, legumes, and vegetable oils. Stanol esters are often added to rapeseed oil based margarine or other foods for its health benefits, including the reduction of LDL cholesterol in blood. By way of non-limiting example, a stanol ester is marketed by the Raisio Group under the trade name Benecol. Sterol esters compounds have the same effect as stanol esters on LDL, but they are partially absorbed by the body. The effects of higher serum plant sterol levels are so far not completely understood.

As used in the specification and claims, the term “Omega-3 fatty acid compound” is intended to include Omega-3 fatty acids (also known as ω-3 fatty acids or n-3 fatty acids), including those found naturally occurring in marine and plant oils, as well as Omega-3 fatty acid precursors and Omega-3 fatty acid derivatives. Omega-3 fatty acids are polyunsaturated fatty acids having an acid end (COOH) and a methyl end (CH₃), with a first double bond between the third and fourth carbon atoms from the methyl end of the carbon chain. Omega-3 fatty acids may have health benefits and are considered essential fatty acids, meaning that they cannot be synthesized by the human body but are vital for normal metabolism. Examples of Omega-3 fatty acids include eicosapentaenoic acid (EPA), docosahexaenoic acid (DHA) and α-linolenic acid (ALA). Common sources of Omega-3 fatty acids include fish oils, algal oil, squid oil and some plant oils such as echium oil and flaxseed oil.

As used in the specification and claims, the term “oral delivery form” is intended to include all known forms and/or techniques in which a composition can be delivered into a patient's body via the patient's mouth. Examples of oral delivery forms include, but are not limited to, pills and tablets, capsules containing powder, capsules containing time-release beads, capsules containing liquid, lozenges, and liquids.

Provided herein are nutritional compositions, regimens and methods for the prevention, mitigation, or treatment of ED. Compositions, regimens and methods of the present invention can be characterized by their ability to prevent, mitigate, or treat ED by providing acute vasodilatory effects while also treating potential long-term causes of ED such as cholesterol-induced narrowing of the blood vessels. Embodiments of the present invention recognize that blood vessel narrowing is often mediated by inflammatory processes and that treatment with niacin compound, in combination with an Omega-3 fatty compound and a phytosterol compound, may have a synergistic anti-inflammatory effect while also promoting cholesterol-related vascular health, both of which provide beneficial effects on ED.

Each embodiment of the compositions, regimens and methods of the present invention involves a combination of any of the three ingredients of a niacin compound, an Omega-3 fatty acid compound, and a phytosterol compound. Some compositions, regimens, and methods of the present invention can be characterized by the absence of any other drug indicated for treatment of ED. Some compositions, regimens and methods of the present invention can also be characterized by the absence of any drug indicated for the treatment of cholesterol. It is also expected that the novel regimens involving combinations of phytosterol compounds and/or Omega-3 fatty acid compounds will reduce the side effects of niacin, including flushing associated with administration of niacin alone, and/or provide beneficial effects on patients' ED with a lesser dose of niacin than if administered alone.

In certain embodiments of the present invention, nutritional regimens of combinations of any of niacin compounds, Omega-3 fatty acid compounds, and phytosterol compounds are prepared and administered to a male patient at preselected times, and in preselected amounts so as to render a benefit to the subject that prevents, mitigates, or treats ED. In one example, the benefit rendered is at least one of acute vasodilatory effects, lowered total cholesterol, altering of the ratio of HDL to LDL, or temporary adjustment of the ratio of HDL to LDL in general, or at particular times, such as at a time before intercourse or other sexual activity is anticipated by a patient.

Prior to treating a patient in accordance with embodiments of the present invention, ED can be diagnosed and evaluated using a variety of techniques, including techniques that are designed to discover potential psychological and physical causes, as well as measure the extent of the patient's ED. Example techniques include the use of patient questionnaires, nocturnal penile tumescence (NPT) testing to confirm whether the patient is achieving erections during sleep, and intracavernosal injection and Doppler ultrasound testing to analyze penile blood flow and determine the extent to which the patient can physically achieve an erection. In addition, cholesterol tests can be performed to determine whether unhealthy cholesterol levels are present and therefore cholesterol-induced blood vessel narrowing may potentially be a contributing factor to the patient's ED. For example, a non-fasting cholesterol test can be used to measure the patient's total cholesterol and HDL cholesterol; a fasting cholesterol test, called a lipid profile or a lipoprotein analysis, can be used to measure the patient's LDL, HDL, total cholesterol, and triglycerides. In practice, a non-fasting test may be performed first to determine if there is a potential problem (e.g., high total cholesterol, low HDL, high total cholesterol/HDL ratio) and if there is a potential problem, then the fasting test may be performed.

FIG. 2 shows dosage ranges for a niacin compound, phytosterol compound, and Omega-3 fatty acid compound that can serve as a guideline when preparing and administering regimens in accordance with an embodiment of the present invention. Dosage ranges can be based generally on the safety and efficacy of ingredients. The dosage ranges shown in FIG. 2 are intended to be exemplary and are not intended to limit the scope of the invention. As discussed later in this specification, the dosage range of any of the ingredients in a composition may be increased or decreased, depending on an individual patient's response and tolerance. For example, the dosage range for the niacin compound could be from 500 mg/day to 1500 mg/day instead of the range shown in FIG. 2.

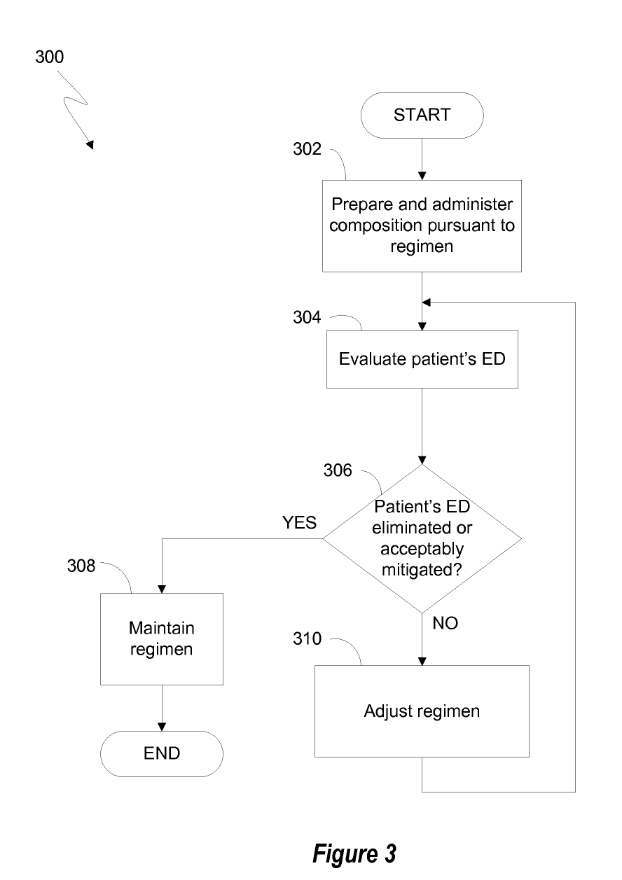

FIG. 3 illustrates an example flowchart for determining an appropriate regimen to prevent, mitigate, or treat ED in a male patient in accordance with an embodiment of the present invention. First, a composition of at least two of the following three ingredients is prepared and administered to the patient in a specified dosage pursuant to a specified regimen: a niacin compound; an Omega-3 fatty acid compound; and a phytosterol compound 302. Although the invention contemplates compounds comprising two of these three ingredients, it is preferable that all three ingredients be included in the compound because combinations of all three ingredients are believed to have a synergistic effect when used to treat ED. Depending on the particular regimen and the specified dosage of the composition, the composition may be repeatedly administered to the patient at certain intervals and for certain durations of time (e.g., administered daily for one week, or administered three times per day for one week).

In an exemplary embodiment, the individual ingredients of the composition, the specified dosage of the composition and the manner of administration are selected on the basis of having the greatest efficacy in preventing, mitigating, or treating ED for the largest number of men, as determined during one or more clinical studies. In an exemplary embodiment, the ingredients and specified dosage of the composition are developed such that the composition can be administered to a wide population of men in an oral delivery form (e.g., a tablet, capsule, etc.) pursuant to a general regimen. For example, the composition can be prepared in an oral delivery form comprising between 500 mg and 1000 mg of a niacin compound and amounts of a phytosterol compound and/or an Omega-3 fatty acid compound in a 1:2 ratio, respectively (e.g., 500 mg of a niacin compound, 1000 mg of a phytosterol compound and/or 1000 mg of an Omega-3 fatty acid compound).

The ingredients and specified dosage of the composition can also be selected on the basis of the combinatorial and/or synergistic effects of the ingredients, whether in a particular patient or in a group of men as determined during one or more clinical studies. For example, Omega-3 fatty acid compounds and phytosterol compounds may be included in the composition because, even if not necessary for treating a particular patient's ED, they may reduce niacin flushing and/or other side effects while also providing cholesterol-related benefits to the patient. Similarly, for example, it may be desirable to include niacin compounds in the composition because, even if not necessary for providing long-term cholesterol-related benefits for a patient's ED, they may provide short-term, acute benefits such as vasodilatory effects.

At some point after administering the composition to the patient pursuant to the specified regimen, the patient's ED is evaluated 304. As previously discussed, evaluation can include a variety of techniques such as questionnaires, NPT testing, and intracavernosal injection and Doppler ultrasound testing.

A determination is then made as to whether the specified regimen with which the patient is currently being treated has eliminated or acceptably mitigated the patient's ED (e.g., from the viewpoint of the patient) 306. If the patient's ED has been eliminated or acceptably mitigated, then the current regimen is simply maintained 308.

If the patient's ED has not been eliminated or acceptably mitigated, adjustments are made to the regimen 310, after which the patient's ED is again evaluated 304 and the steps repeat. In an exemplary embodiment, the regimen is adjusted by increasing the dosage of the composition (i.e., proportionately increasing the dosage of each ingredient in the composition) as necessary to eliminate or acceptably mitigate the patient's ED, but only up to a specified dosage limit. For example, the specified dosage limit can be based on any one of the ingredients of the composition exceeding the amounts specified in FIG. 2. If the patient's ED has not been eliminated or has not been acceptably mitigated after increasing the dosage of the composition up to the specified dose limit, then other treatment options must be considered.

In other embodiments, in the event the patient's ED has not been eliminated or has not been acceptably mitigated after increasing the dosage of the composition up to the specified dose limit, adjustments to the regimen can be made by adding and/or removing ingredients from the composition and adjusting amounts of ingredients individually. If no combination of individual ingredients or amounts of individual ingredients eliminates or acceptably mitigates the patient's ED, then other treatment options are considered.

Applicant has recently commenced clinical studies to further explore the inventive concepts herein. Among other things, Applicant has designed a human clinical trial protocol for fuller evaluation of the effect of administering the regimens and compositions described herein to men having ED. The above-described regimens are illustrative and can be adjusted in accordance with the results obtained from the clinical studies, such as a finding of greater efficacy of a particular ingredient in providing short-term and/or long-term ameliorative effects on ED informing a decision to increase the amount of that particular ingredient in the composition that is administered to all patients.

Advantages of the regimens and methods disclosed herein include fewer side effects than might otherwise be present with current ED and/or cholesterol medications such as statins, whether by virtue of replacing such current drugs with the regimens disclosed herein and/or by the use of a lower dose of such current drugs in combination with the regimens and methods herein.

Still further advantages include a shorter time to provide positive effects on cholesterol and ED based upon the surprising synergy of niacin compounds, Omega-3 fatty acid compounds, and phytosterol compounds in controlling cholesterol and in mitigating or treating ED.

### Example

The following is an example of a regimen to prevent, mitigate, or treat ED in a male patient in accordance with an embodiment of the present invention. A composition is prepared in capsule form (or some other oral delivery form) comprising 100 mg of nicotinic acid, 200 mg of phytosterol compounds, and 200 mg of Omega-3 fatty acid compounds (100 mg of EPA and 100 mg of DHA). One capsule per day is administered to the patient for one week, after which the patient's ED is evaluated. If the patient's ED is eliminated or acceptably mitigated, the regimen is maintained. If the patient's ED is not eliminated or acceptably mitigated, the dosage is increased to three capsules per day for one week (i.e., a total daily dosage of 300 mg of nicotinic acid, 600 mg of phytosterol compounds, and 600 mg of Omega-3 fatty acid compounds). Alternatively, one or more capsules containing increased amounts of the ingredients can be administered to achieve these same total daily dosages of the ingredients.

Again, the patient's ED is evaluated, and if the patient's ED is eliminated or acceptably mitigated, the regimen is maintained. If the patient's ED is not eliminated or acceptably mitigated, the dosage of the composition is increased by administering five capsules per day for two weeks (i.e., a total daily dosage of 500 mg of nicotinic acid, 1000 mg of phytosterol compounds, and 1000 mg of Omega-3 fatty acid compounds). Alternatively, one or more capsules containing increased amounts of the ingredients can be administered to achieve these same total daily dosages of the ingredients. For example, one capsule can contain between 100 mg and 500 mg of nicotinic acid and proportionately increased amounts of phytosterol compounds and Omega-3 fatty acid compounds.

Again, the patient's ED is evaluated, and if the patient's ED is eliminated or acceptably mitigated, the regimen is maintained. If the patient's ED is not eliminated or acceptably mitigated, the dosage of the composition is increased by administering 10 capsules per day for one week (i.e., a total daily dosage of 1000 mg of nicotinic acid, 1200 mg of phytosterol compounds, and 1200 mg of Omega-3 fatty acid compounds). Alternatively, one or more capsules containing increased amounts of the ingredients can be administered to achieve these same total daily dosages of the ingredients. For example, one capsule can contain between 100 mg and 1000 mg of nicotinic acid and proportionately increased amounts of phytosterol compounds and Omega-3 fatty acid compounds.

Lastly, the patient's ED is evaluated, and if the patient's ED is eliminated or acceptably mitigated, the regimen is maintained. If the patient's ED is still not eliminated or acceptably mitigated, then other treatment options must be considered.

While the principles of the invention have been described above in connection with preferred embodiments, it is to be clearly understood that this description is made only by way of example and not as a limitation of the scope of the invention.

1. A composition for the prevention, mitigation, or treatment of erectile dysfunction, the composition comprising:
at least two members of the group consisting of a niacin compound, an Omega-3 fatty acid compound, and a phytosterol compound.

2. The composition of claim 1, wherein the composition comprises a niacin compound, an Omega-3 fatty acid compound, and a phytosterol compound.

3. The composition of claim 2, wherein the composition comprises between 100 mg and 500 mg of the niacin compound, an Omega-3 fatty acid compound, and a phytosterol compound.

4. The composition of claim 1, wherein the composition comprises inositol hexanicotinate.

5. The composition of claim 1, wherein the at least two members consist of a niacin compound and one member of the group consisting of an Omega-3 fatty acid compound and a phytosterol compound.

6. The composition of claim 5, wherein the niacin compound and the one member of the group consisting of an Omega-3 fatty acid compound and a phytosterol are present in the composition in a 1:2 ratio, respectively.

7. The composition of claim 1, wherein the composition does not contain any statin or other cholesterol modifying pharmaceutical.

8. The composition of claim 1, wherein the composition does not contain any additional pharmaceutical intended to treat erectile dysfunction.

9. The composition of claim 1, wherein the composition is in an oral delivery form.

10. A method of preventing, mitigating, or treating erectile dysfunction in a human male, the method comprising the steps of:
preparing a first composition comprising at least two members of the group consisting of a niacin compound, an Omega-3 fatty acid compound, and a phytosterol compound; and
administering the first composition to the human male in a first dosage.

11. The method of claim 10, wherein the first composition comprises a niacin compound, an Omega-3 fatty acid compound, and a phytosterol compound.

12. The method of claim 10, wherein the at least two members consist of a niacin compound and one member of the group consisting of an Omega-3 fatty acid compound and a phytosterol compound.

13. The method of claim 12, wherein the niacin compound and the one member of the group consisting of an Omega-3 fatty acid compound and a phytosterol are present in the first composition in a 1:2 ratio, respectively.

14. The method of claim 10, further comprising the steps of:
measuring a cholesterol level in the human male; and
determining that at least one compound in the first composition will positively affect the measured cholesterol level in the human male.

15. The method of claim 10, further comprising the step of:
administering the first composition to the human male in a second dosage, wherein the second dosage is greater than the first dosage.

16. The method of claim 10, wherein the first composition does not contain any statin or other cholesterol modifying pharmaceutical.

17. The method of claim 10, wherein the first composition does not contain any additional pharmaceutical intended to treat erectile dysfunction.

18. The method of claim 10, wherein the first composition is administered to the human male in an oral delivery form.

19. The method of claim 10, wherein the first composition comprises inositol hexanicotinate.
